(19) United States
(12) Patent Application Publication
(10) Pub. No.: US 2014/0378927 A1
(43) Pub. Date: Dec. 25, 2014

(54) NURSING BRA PAD AND PAD HOLDER

(71) Applicant: Mary Burket, Dedham, MA (US)

(72) Inventor: Mary Burket, Dedham, MA (US)

(21) Appl. No.: 13/923,478

(22) Filed: Jun. 21, 2013

(51) Int. Cl. A61F 13/14

(52) U.S. Cl. CPC A61F 13/141
USPC 604/368; 604/388

(57) ABSTRACT

A nursing pad and nursing pad holder configured to be attached by a band to a bra strap. The pad and pad holder may be oriented over a nipple of a nursing mother, between the breast and bra cup, thereby absorbing any leaking milk before or after nursing.

### BACKGROUND OF THE INVENTION

1. Field of the Invention

The present invention relates generally to devices designed to facilitate the process of nursing an infant. More particularly, the present invention relates to a device that may attach to a bra that may absorb leaking breast milk.

2. Description of Related Art

For many years, women have had trouble finding clothing suitable for wear when nursing babies. Particularly problematic, are nursing pads, which are designed to absorb and catch leaking breast milk. Prior art nursing pad are often cumbersome, bunch easily, can be lost or dropped, and can often be seen through a bra.

The nursing pads of the prior art generally are crumpled or folded when the bra cup is folded or adjusted for nursing. This creates lumps in the nursing pad which will then have to be smoothed before replacing the bra cup. If the lumps are not removed, the pad will create an unsightly silhouette in the bra. Further still, many of these nursing pads are fully removable, causing them to be easily lost, and difficult to handle during nursing, as well as causing a number of sanitary issues by being placed on various surfaces.

What many women want is something comfortable and attractive, yet convenient enough so that it can be quickly and easily removed when actually nursing that may be conveniently replaceable and not be easily lost.

Therefore, what is needed is a nursing pad device that may be attached to a bra, but be easily removable when needed without the fear of losing, dropping, or otherwise misplacing the pad.

### SUMMARY OF THE INVENTION

The subject matter of this application may involve, in some cases, interrelated products, alternative solutions to a particular problem, and/or a plurality of different uses of a single system or article.

In one aspect, a nursing pad holder is provided. The holder has a top and bottom section joined together at the outer edges. The top and bottom form a holding region accessible by an aperture defined by the bottom. A pad may be disposed within the holding region. The pad may enter and exit the holding region through the aperture formed by the bottom. A band is attached to the holder and is configured to allow the device to be removably attached to a bra strap of the user.

In another aspect, a nursing pad device is provided. The device comprises an absorbent pad sized and configured to cover a human nipple. A band is attached to the pad at a proximal end, and attached by an attachment region at a distal end to a bra strap of a bra. When the bra is attached to the user, the pad may be positioned over a nipple of the user, and held in place by a friction between a breast of the user and a cup of the bra.

### BRIEF DESCRIPTION OF THE DRAWINGS

FIG. 1 provides a top perspective view of an embodiment of the nursing pad holder.

FIG. 2 provides a bottom perspective view of an embodiment of the nursing pad holder.

FIG. 3 provides a front view of an embodiment of the nursing pad holder.

FIG. 4 provides a rear view of an embodiment of the nursing pad holder.

FIG. 5 provides a perspective view of an embodiment of the pad.

FIG. 6 provides a perspective view of an embodiment of the pad.

FIG. 7 provides a view of an embodiment of the pad and pad holder in use.

FIG. 8 provides a view of an embodiment of the pad and pad holder in use.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Generally, the present invention concerns a pad configured to be placed in a bra to absorb milk from a nipple of a nursing human mother. The pad may be attachable to a bra of the mother by attachment to the bra cup, bra strap, or the like. A pad holder may allow the pad to be removably stored therein, with the pad holder being attached to the bra. In many embodiments, the pad may be washable and re-usable, though in other embodiments the pad may be configured to be disposable.

The pad may be generally circular in shape to match a shape of a woman's nipple. However, it should be understood that the pad may be of any shape capable of covering a woman's nipple. The pad may further be configured to absorb an amount of fluid that may leak from a woman's breast throughout the course of the day. This leaking may come from immediately after nursing, before nursing, or generally throughout the day or night. The pad may be made of any material capable of absorbing this fluid. Examples of materials of which the pad may be made include cotton, terry cloth, polyester, and similar fabrics. In a particular embodiment, the pad may be formed of a thin outer fabric with an absorbent material interior. Examples of absorbent material interiors may include absorbent fabrics, absorbent foams, sponges, absorbent gel, and the like.

The pad or pad holder may be attached to the bra in any manner capable of holding the pad in place over the nipple while the bra is in place over the woman's breast. Further, in many embodiments, either the pad or pad holder may comprise a band such as a string, tether, ribbon, strap or other attachment mechanism that allows the pad to be removed from over the nipple for nursing while remaining attached to the bra, and easily replaced in position once nursing concludes. In one embodiment, the pad may have a band attached, the band being attachable to a bra strap. In another embodiment, the pad may be placed in a pad holder, the pad holder having a string attachable to the bra strap. In yet another embodiment, the pad and/or pad holder may be directly attachable to the cup of the bra over where a nipple would be located.

The pad may be held in position over the nipple when the woman is not nursing. This may be achieved in any manner capable of holding the pad substantially in position, such as by friction, by removable attachment of the pad to the bra, or by guides such as protrusions or detents formed into the bra cup to urge the pad into proper position, among others.

In one embodiment, the band may be adjustable in length, allowing a user to adjust its sizing depending on user's size.

The nursing pad and nursing pad holder contemplated herein provide a number of advantages over the prior art. One such advantage is the ability to absorb leaking breast milk in a manner that is convenient, comfortable, and not observable to others. Another advantage is provided by the pad or pad holder attaching, via a band, to a bra strap of the user. This allows the user to remove the pad when nursing and to easily retrieve it once the nursing is finished. Further, the device may be used with any type of bra, including a traditional bra, sports bra, nursing bra, and the like. The bras usable with the device may be specifically configured for its use, or may be standard bras not configured in any particular way.

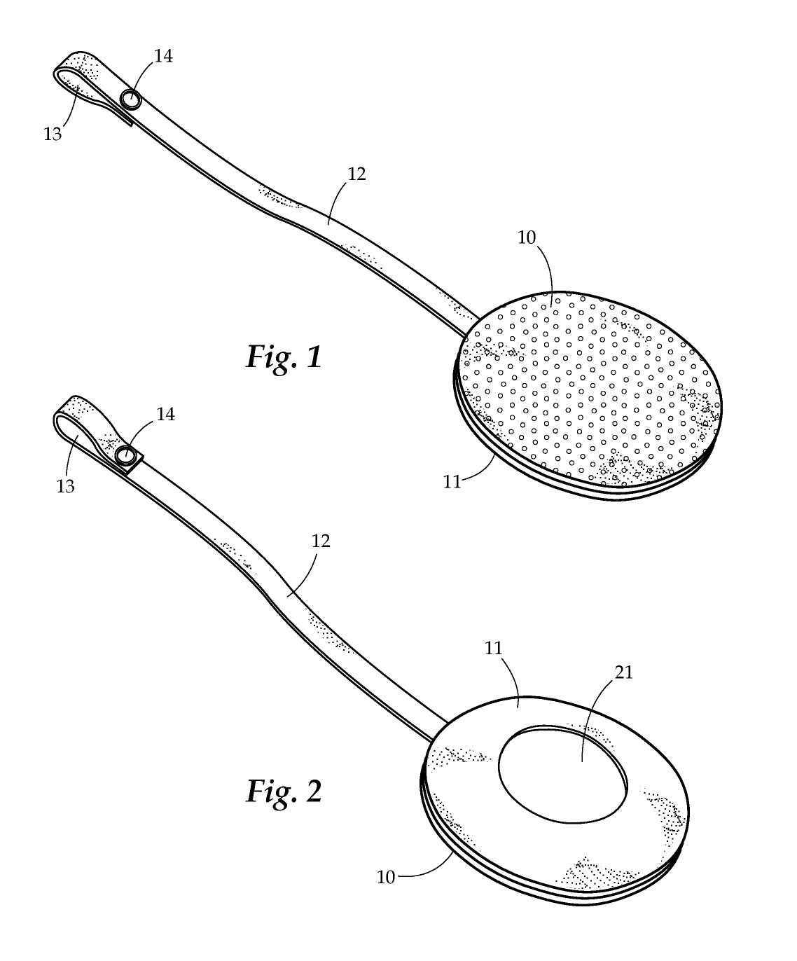

Turning now to FIG. 1, an embodiment of the nursing pad is provided. In this embodiment, the pad (not shown) is stored within a pad holder having a top surface 10 and bottom surface 11 forming a holding region configured for removable storage of the pad. A band 12 is connected to the top surface 10, bottom surface 11, or both. The band 12 forms an attachment region 13 which folds over and is held in place by a snap 14. The attachment region 13 may be held in place by any mechanism capable of holing it in a looped form that allows the attachment region 13 to connect to a bra strap.

It should be understood that the attachment region may be any structure that allows the band to be attached to the bra. For example, in another embodiment, the attachment region 13 may be tightenable to snugly hold a bra strap. In yet another embodiment, the band may directly attach to the strap by, for example, a clip, snap, or the like.

FIG. 2 shows a bottom view of an embodiment of the nursing pad. In this view the pad 21 can be seen held in place by the pad holder top 10 and bottom 11. The pad 21 is flexible and can be removed through the aperture defined by the bottom 11 of the pad hold holder. A band 12 is connected to the top surface 10, bottom surface 11, or both. The band 12 forms an attachment region 13 which folds over and is held in place by a snap 14. As noted previously, the attachment region 13 may be held in place by any mechanism capable of holing it in a looped form that allows the attachment region 13 to connect to a bra strap, and optionally that may allow tightening of the attachment region 13 about a bra strap.

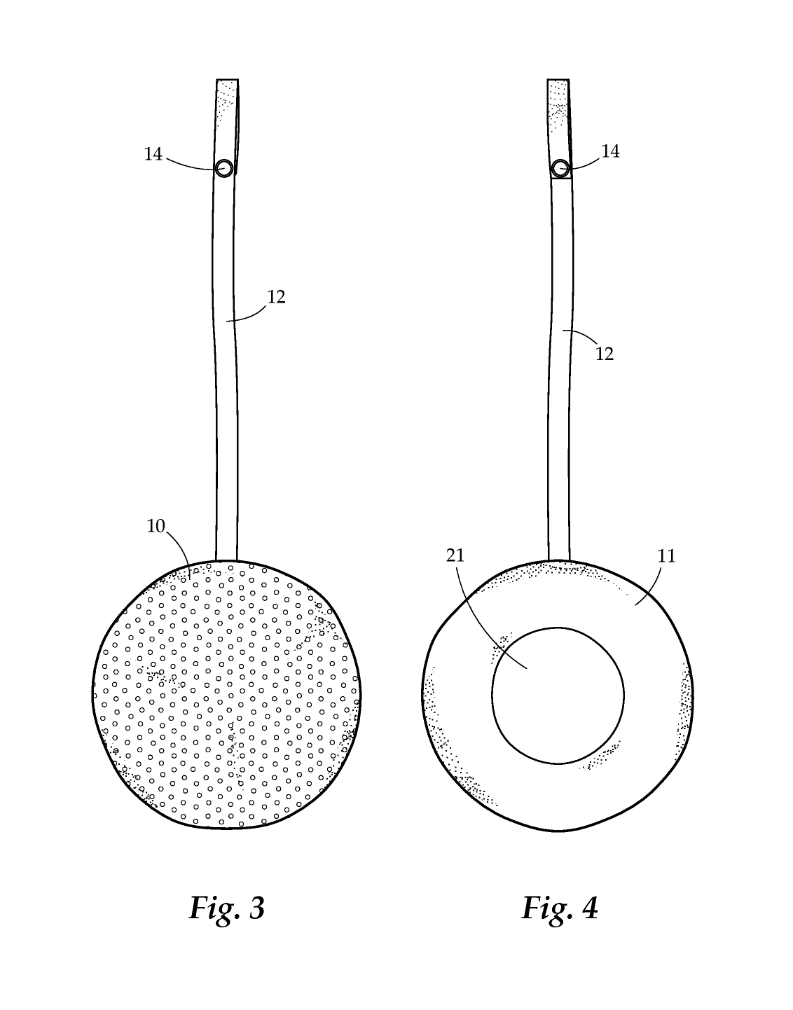

FIGS. 3 and 4 provides front and rear views of embodiments of the nursing pad. The pad 21 can be seen held in place by the pad holder top 10 and bottom 11. The pad 21 is flexible and can be removed through the aperture defined by the bottom 11 of the pad hold holder. A band 12 is connected to the top surface 10, bottom surface 11, or both. The band 12 forms an attachment region (not shown) which folds over and is held in place by a snap 14.

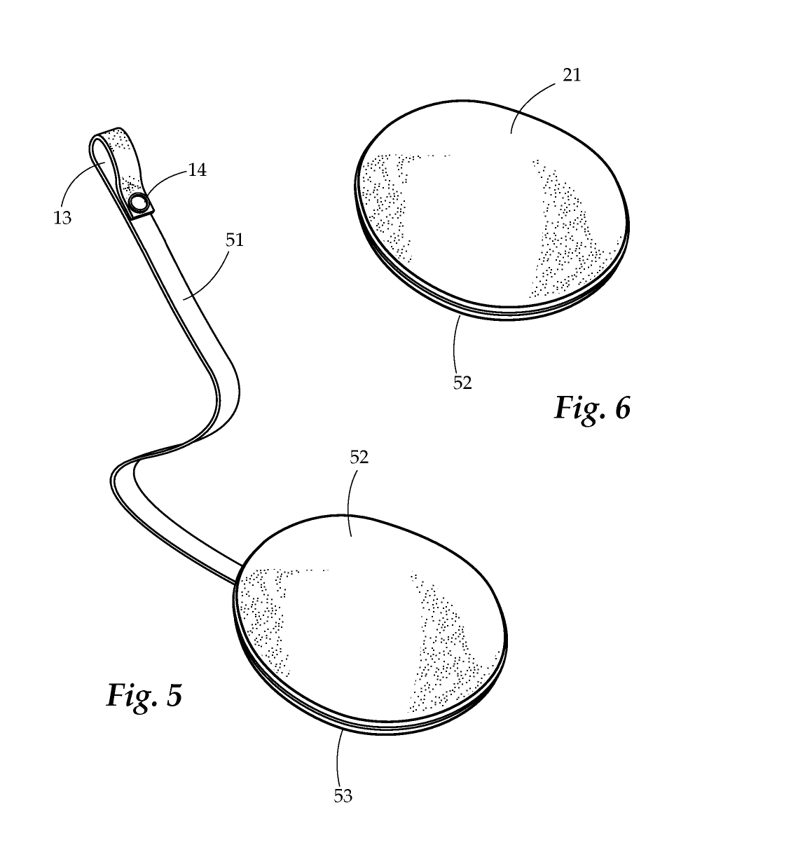

FIG. 5 provides a view of another embodiment of the nursing pad. In this embodiment, the pad 52 is directly attached to a band 51. In this embodiment, the band 51 may be attached to the bra strap (not shown), similarly to the structure of band 12. This embodiment allows the pad itself to be directly oriented over a nipple when the bra cup is in place, attached to the bra, and easily moved aside when the woman is nursing. The pad 52 may be formed of any material, as noted above. In this embodiment, a stitching 53 extends about the perimeter of the pad 52.

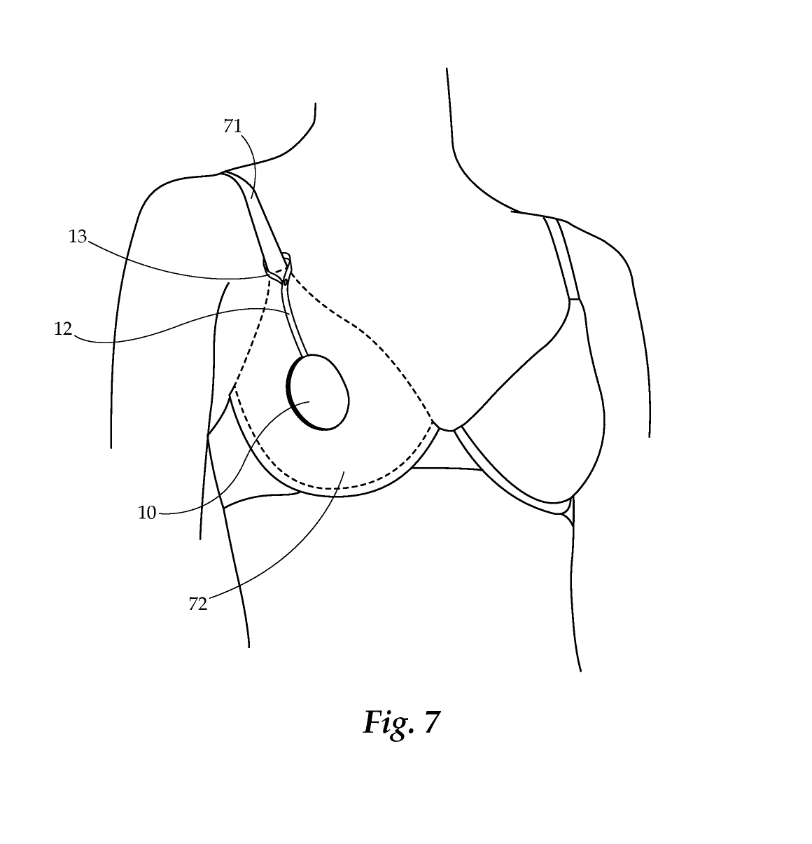

FIG. 6 provides a view of an embodiment of a pad 21. The pad 21 may fit into the pad holder 10, 11. Alternatively, the pad 21 may be held by friction between a nipple and bra cup. Or further alternatively, the pad 21 may be attached to an interior of the bra by a band that is permanently attached to the bra and snaps onto a pad. In this embodiment, the pad or pad holder may be removed from the band, the band being integrated into the bra.

FIG. 7 provides an embodiment of the nursing pad in use. In this embodiment, the nursing mother is wearing a traditional bra. The band 12 is attached, via that attachment region 13, to bra strap 71. The pad holder 10 (though it could be a pad directly) is oriented over a nipple of the mother. The bra cup 72 is shown as a partial cutaway. The cup 72 holds the pad holder 10 in place by urging against the breast of the user. In some embodiments, physical guides may urge the pad or pad holder into place over the nipple.

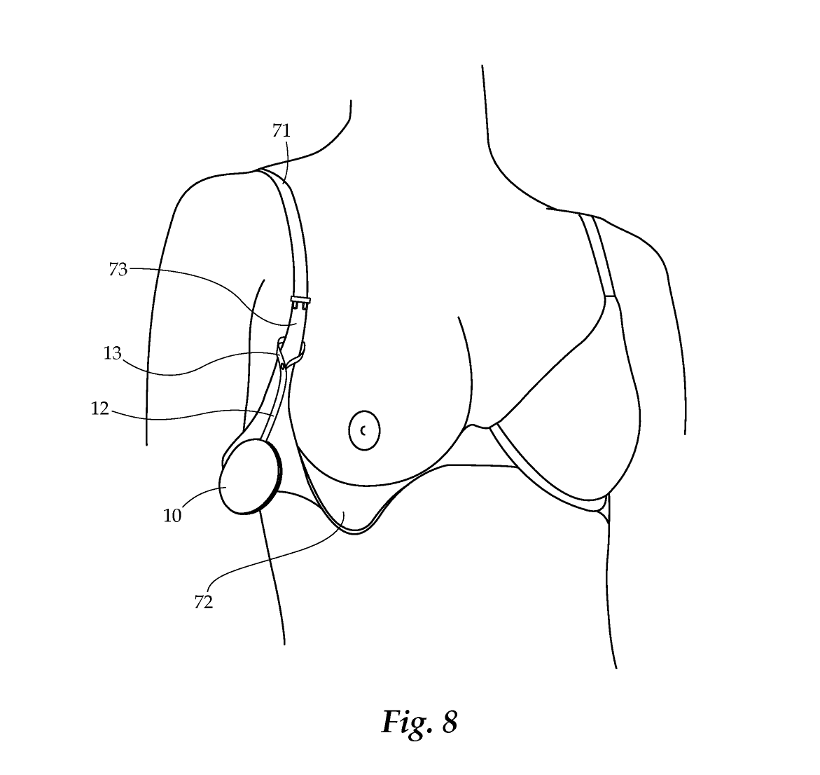

FIG. 8 provides an embodiment of the nursing pad in use. In this embodiment, the nursing mother is wearing a nursing bra. The bra strap has two portions, the traditional strap 17 and the secondary strap 73 that allows for the nursing bra to function. The secondary strap sits under the cup when the cup is attached—in a non-nursing mode. The cup is partially removable and may be folded down in a nursing mode, exposing the secondary strap, which holds the bra up over the woman's shoulder. Here, the attachment region 13 is attached to the secondary strap 73. The mother is preparing to nurse and therefore has removed the pad holder 10 from over the nipple, and has pulled the cup 72 downward. Once nursing is finished, the pad holder 10 may be conveniently retrieved because it is attached to the strap 73. The pad holder 10 or pad (not shown) may be oriented over the nipple, and the cup 72 returned to a non-nursing position.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A nursing pad device comprising:
a holder, the holder having a top and bottom joined together at their outer edges, the top and bottom forming a holding region of the holder, the bottom defining an aperture, the aperture providing access to the holding region;
a pad disposed within the holding region, the pad removable through the aperture;
a band attached to the holder at a proximal end, the band having an attachment region at a distal end allowing the band to be removably attached to a bra strap of a bra.

2. The nursing pad device of claim 1 wherein the bra is attached to a user, the attachment region of the band being attached to the bra strap of the bra, and wherein the holder is positioned such that the aperture of the bottom is oriented over a nipple of the user providing direct contact between the nipple and the pad.

3. The nursing pad device of claim 2 wherein the bra is a nursing bra, and wherein the band attachment region is attached to a secondary strap of the nursing bra.

4. The nursing pad device of claim 1 wherein the pad is washable.

5. The nursing pad device of claim 1 wherein the pad is disposable.

6. The nursing pad device of claim 1 wherein the pad further comprises an interior region comprised of an absorbent material.

7. The nursing pad device of claim 6 wherein the absorbent material is an absorbent gel.

8. The nursing pad device of claim 1 wherein the attachment region is formed by a snap mechanism on the band.

9. The nursing pad device of claim 2 wherein the attachment region includes a tightening mechanism allowing the attachment region to be tightened about the bra strap.

10. The nursing pad device of claim 2 wherein the holder is positioned, and held in the position by a friction between a breast of the user and a cup of the bra.

11. A nursing pad device comprising:
a bra;
an absorbent pad sized and configured to cover a human nipple;
a band attached to the pad at a proximal end, the band having an attachment region at a distal end allowing the band to be removably attached to a bra strap of the bra;
wherein the bra is attached to a user, the attachment region of the band being attached to the bra strap; and
wherein the pad is positioned such that it is oriented over a nipple of the user.

12. The nursing pad device of claim 11 wherein the bra is a nursing bra, and wherein the band attachment region is attached to a secondary strap of the nursing bra.

13. The nursing pad device of claim 11 wherein the pad is washable.

14. The nursing pad device of claim 11 wherein the pad is disposable.

15. The nursing pad device of claim 11 wherein the pad further comprises an interior region comprised of an absorbent material.

16. The nursing pad device of claim 15 wherein the absorbent material is an absorbent gel.

17. The nursing pad device of claim 11 wherein the attachment region is formed by a snap mechanism on the band.

18. The nursing pad device of claim 11 wherein the attachment region includes a tightening mechanism allowing the attachment region to be tightened about the bra strap.

19. The nursing pad device of claim 11 wherein the pad is positioned and held in position by a friction between a breast of the user and a cup of the bra.

20. The nursing pad device of claim 11 wherein a length of the band is adjustable.
